(19) United States
(12) Patent Application Publication
(10) Pub. No.: US 2020/0385331 A1
(43) Pub. Date: Dec. 10, 2020

(54) ALTERNATIVE KETONE ESTERS AND PRODUCTION PROCESSES THEREOF

(71) Applicant: KetoneAid Inc., Falls Church, VA (US)

(72) Inventor: Frank Borges LLOSA, Falls Church, VA (US)

(21) Appl. No.: 16/736,136

(22) Filed: Jan. 7, 2020

Related U.S. Application Data
(63) Continuation of application No. 16/408,424, filed on May 9, 2019, now abandoned.
(60) Provisional application No. 62/669,350, filed on May 9, 2018.

(51) Int. Cl. C07C 69/675; C07C 67/31

(52) U.S. Cl. CPC C07C 69/675; C07C 67/31

(57) ABSTRACT

Embodiments described herein provide for therapeutic compounds related to alternative ketone esters, and production processes thereof.

### CROSS-REFERENCE TO RELATED APPLICATIONS

This applications claims priority to U.S. Provisional Application 62/669,350 filed on May 9, 2018, and entitled “ALTERNATIVE KETONE ESTERS AND PRODUCTION PROCESS THEREOF” the disclosure of which is incorporated in its entirety herein.

### FIELD

The embodiments provided herein generally relate to therapeutic compounds and production processes thereof.

### BACKGROUND

The present invention generally relates to means of protecting or stabilizing the beta hydroxy group in β-hydroxybutyrate in order to prevent beta hydroxy dehydration in the presence of strong acid, such as found in the stomach. That reaction is a type of gastric degradation which yield compounds with no known metabolic therapy. The goal in protecting or stabilizing the compounds is to mitigate loss of the less stable hydroxy group and the formation of a double bond between the 2nd and 3rd carbons such that when administered orally, dehydration via gastric degradation is reduced resulting in increased serum levels of 3-hydroxybutyrate on a gram to gram basis thus increased efficacy within the body. Stabilizing the beta hydroxy group is possible by replacing the carboxylic acid functional group with an ester functional group, which prevents resonance between the two oxygen atoms by hydrogen and considerably stabilizes the beta hydroxy group reducing dehydration by as much as 40% compared to salts and the free acid of β-hydroxybutyrate. Protecting the beta hydroxy group is defined by replacing the beta hydroxy group with a second molecule of β-hydroxybutyrate forming an ester bond at the previous beta hydroxy functional group and yielding a dimer ester (DE) of β-hydroxybutyrate. Further described herein are similar therapeutic agents, compositions, and production processes thereof.

Ketone bodies are chemical compounds which are produced by the liver from fatty acids released from adipose tissue. Ketone bodies themselves may be used as a source of energy in most tissues of the body, especially in the absence of glucose. The intake of compounds that boosts the levels of ketone bodies in the blood can lead to various clinical benefits. Further, many benefits are correlated with being in ketosis, both through nutrition and supplementation.

The free acid or buffered free acid of β-hydroxybutyrate, β-hydroxybutyrate salt, and especially esters thereof have been shown to be efficacious in treating a variety of conditions, such as fatigue, visual impairments, and diabetes to name a few. Further, cognitive function is shown to be vastly increased. Because of stabilization, the esters have shown to be far more effective at a much relatively smaller dose in comparison to β-hydroxybutyrate and salts thereof. Protected DEs are even less susceptible to beta dehydration.

Commonly, β-hydroxybutyrate is administered orally, as this is the most readily available means of administration for user. Oral consumption requires that the 3-hydroxybutyrate, in all its versions, pass through the gut wherein degradation occurs. Passing through the gut dehydrates the beta-hydroxy group on β-hydroxybutyrate.

Described herein are novel compounds which prevent gut degradation and increase efficacy of β-hydroxybutyrate and esters thereof when consumed orally. Protecting the Beta Hydroxy group such as by forming an ester would increase efficacy. It is also possible to replace the beta hydroxy group with an ester bond on the salts, free acids or esters to increase serum level efficacy. In addition to protecting the beta hydroxy group with β-hydroxybutyrate and forming the Dimer Ester (DE), other organic acid groups such as an acetate group in place of the beta hydroxy group would reduce dehydration. Esterase found outside of the acidic stomach throughout the body would cleave the ester bond after traveling through the gut. The product would yield one molecule of acetic acid for every molecule of β-hydroxybutyrate in order to increase serum β-hydroxybutyrate levels when compared to non-protected salts, free acids or esters of β-hydroxybutyrate. Another option would be to create an ester bond at the beta hydroxy group, substituting, for instance, non-cyclic alcohol sugars or other alcohols for the beta hydroxy group to prevent dehydration in the free acid/salt for oral administration. Naturally occuring esterase would cleave the bond leaving one molecule of alcohol or alcohol sugar such as xylitol or sorbitol for every protected molecule of β-hydroxybutyrate.

### SUMMARY

The embodiments disclosed herein provide for a composition comprising (4R)-hydroxybutyl (3R)-hydroxybutyrate. The composition may further comprise (3R)-hydroxybutyl (3R)-hydroxybutyrate.

In one aspect, the composition may also be comprised of one or more of the following: β-hydroxybutyrate/β-hydroxybutyrate, beta-hydroxybutyrate salts, free acid beta-hydroxybutyrate, 1,3 butanediol, β-hydroxybutyrate/1,3-butanediol/β-hydroxybutyrate, 3-hydroxybutyrate/β-hydroxybutyrate/1,3-butanediol, and MCT oil.

In one aspect, the composition has at least 60% enantiomeric excess of any of the following: (4R)-hydroxybutyl (3R)-hydroxybutyrate, (3R)-hydroxybutyl (3R)-hydroxybutyrate, β-hydroxybutyrate/β-hydroxybutyrate, beta-hydroxybutyrate salts, free acid beta-hydroxybutyrate, 1,3 butanediol, β-hydroxybutyrate/1,3-butanediol/β-hydroxybutyrate, and β-hydroxybutyrate/β-hydroxybutyrate/1,3-butanediol.

In one aspect, a method of production of (4R)-hydroxybutyl (3R)-hydroxybutyrate comprises the steps of protecting a primary alcohol or hydroxy group with a non-reactive protecting group, attaching another molecule at the secondary alcohol or secondary hydroxy group using a silyl group, and deprotecting the primary hydroxy group to make the target molecule.

In one aspect, the method of production yields products having at least 60% enantiomeric excess (4R)-hydroxybutyl (3R)-hydroxybutyrate.

### DETAILED DESCRIPTION

The specific details of the single embodiment or variety of embodiments described herein are set forth in this application. Any specific details of the embodiments are used for demonstration purposes only and no unnecessary limitations or inferences are to be understood therefrom.

A detailed explanation of the composition of matter according to preferred embodiments of the present invention are described below. In general, the invention relates to novel compounds which prevent gut degradation and increase efficacy of β-hydroxybutyrate salts, free acids and esters, or any combination thereof following oral consumption of the foodstuff.

Ketosis is a fat-based metabolism, a state indicated by elevated levels of ketones in the blood and in which a person's body produces ketones for fueling metabolism rather than primarily using glycogen to make glucose. The ketogenic diet, which can initiate and maintain ketosis, was developed initially to treat pediatric refractory epilepsy. The original diet required ingesting calories primarily from fat, with a minimally sufficient amount of proteins to allow for growth and repair, and with a very restricted amount of carbohydrates. A typical diet would include a 4:1 ratio of fat to combined protein and carbohydrate (by weight). The ketogenic diet can allow one's body to consume fats for fuel rather than carbohydrates. Normally, the carbohydrates contained in food are stored as glycogen in the body and then, when needed, converted into glucose. Glucose is particularly important in fueling brain-function however during the peak of ketosis, up to 70% of the total calories utilized by the brain can be derived from ketones. During times of starvation or extreme carbohydrate fasting, bodily protein is converted into glucose via gluconeogenesis to provide the deficit glucose for the brain.

When a body lacks carbohydrates, the liver converts fat into fatty acids and ketone bodies. The ketone bodies pass into the brain and replace glucose as an energy source. An elevated level of ketone bodies in the blood, i.e. ketosis, has been shown to reduce the frequency of epileptic seizures. Ketosis may also improve brain-function when a person's body cannot properly use glucose, such as in Alzheimer's patients and those with concussions or other brain damage including age-related glucose impairment in otherwise healthy adults. The specific cause of “Age related dementia” has not been identified to be caused by a specific vector. Evidence suggests it has to do with the same pyruvate dehydrogenase mitochondrial impairment that is so obvious in diseased and traumatized brains. We believe all adults over 35 suffer from it to one degree or another and would thus benefit from ketone therapy.

In addition to improved brain-function, ketones can improve muscle performance, such as in endurance athletes. This is because the body can only store and convert about 100-minutes' worth of glycogen into useful glucose during extreme and prolonged exercise, such as in bicycle races and long-distance running. Athletes can train to extend their body's capacity, but there are limits. Yet, with a second or alternative source of energy, from ketones, the body can continue to perform beyond the individual's capacity to produce glucose. Further, studies have shown that ketones can improve endurance performance by as much as four percent due in part to the glycogen sparing effect of the additional fuel.

The compounds of the invention as defined above reduces plasma levels of fatty acids. A compound of the invention may therefore be used to reduce the level of free fatty acids circulating in the plasma of a subject. As such it may be used to treat a condition which is caused by, exacerbated by or associated with elevated plasma levels of free fatty acids in a human or animal subject. A human or animal subject may therefore be treated by a method which comprises the administration thereto of a compound of the invention as defined above. The condition of the subject may thereby be improved or ameliorated.

Conditions which are caused by, exacerbated by or associated with elevated plasma levels of free fatty acids include, but are not limited to: neurodegenerative diseases or disorders, for instance Alzheimer's disease, Parkinson's disease, Huntington's chorea; hypoxic states, for instance angina pectoris, extreme physical exertion, intermittent claudication, hypoxia, stroke and myocardial infarction; insulin resistant states, for instance infection, stress, obesity, diabetes and heart failure; and inflammatory states including infection and autoimmune disease.

The results of studies using a hypertrophied mouse heart model indicate that the hypertrophied and failing heart shifts to ketone bodies as a significant fuel source for oxidative ATP production. Heart disease is characterized by a decreased ability to oxidize fatty acids leaving the diseased heart fuel starved. Specific metabolite biosignatures of in vivo cardiac ketone utilization were identified.

In addition to reducing plasma levels of fatty acids, a compound of the invention acts on the appetite centres in the brain. In particular, a compound of the invention increases the levels of various anorexigenic neuropeptides (neuropeptides known to be associated with decreased food intake and decreased appetite) in the appetite centres of the brain and also induces higher levels of malonyl CoA, a metabolite associated with decreased appetite and food intake. The invention therefore further provides a compound of the invention as defined above for use in treating a condition where weight loss or weight gain is implicated. For example, the compound may be used in suppressing appetite, treating obesity, promoting weight loss, maintaining a healthy weight or decreasing the ratio of fat to lean muscle in a subject. The subject in each case may be a healthy subject or a compromised subject. A healthy subject may be, for instance, an individual of healthy weight for whom physical performance and/or physical appearance is important. Examples include members of the military, athletes, bodybuilders and fashion models, A compromised subject may be an individual of non-healthy weight, for instance an individual who is overweight, clinically obese or clinically very obese. A compromised subject may alternatively be an individual of healthy or unhealthy weight who is suffering from a clinical Condition, for instance a condition listed below.

An individual of healthy weight has a body mass index (BMI) of 18.5 to 24.9; an individual who is overweight has a body mass index (BMI) of from 25 to 29.9; an individual who is clinically obese has a body mass index of from 30 to 39.9; and an individual who is clinically very obese has a body mass index of 40 or more.

In addition to reducing plasma levels of fatty acids and acting on the appetite centre in the brain, a compound of the invention increases brain metabolic efficiency, by increasing brain phosphorylation potential and the AG of ATP hydrolysis. A compound of the invention thereby promotes improved cognitive function and can be used to treat cognitive dysfunction or to reduce the effects of neurodegeneration. A compound of the invention also increases the level of the neuropeptide Brain Derived Neurotrophic Factor (BDNF) in both the paraventricular nucleus (the appetite centre of the brain) and the hippocampus (a part of the brain known to be important for memory). As well as decreasing appetite, BDNF is known to prevent apoptosis and promote neuronal growth in basal ganglia and other areas of interest, thus the increased levels of BDNF produced by the compound of the invention are expected to inhibit neurodegeneration, limit neural tissue death after hypoxia or trauma and promote neural tissue growth.

A compound of the invention also increases the level of the anorexigenic neuropeptide Cocaine-and-Amphetamine Responsive Transcript (CART). CART is known to promote alertness as well as to decrease appetite. Thus, the increased levels of CART produced b the compound of the invention are expected to improve cognitive function.

The compounds of the invention are therefore useful for (a) promoting alertness and improved cognitive function, and (h) inhibiting neurodegeneration. The invention therefore further provides a compound of the invention as defined above for use in promoting alertness or improving cognitive function, or in treating cognitive dysfunction.

The invention also provides a compound of the invention as defined above for use in treating, preventing, or reducing the effects of, neurodegeneration, free radical toxicity, hypoxic conditions or hyperglycaemia.

In one embodiment, the compound of the invention as defined above is for use in treating, preventing, or reducing the effects of, neurodegeneration. A compound of the invention may be used to treat, prevent, or reduce the effects of neurodegeneration arising from any particular cause. The neurodegeneration may for instance be caused by a neurodegenerative disease or disorder, or may be caused by aging, trauma, anoxia and the like. Examples of neurodegenerative diseases or disorders that can be treated using a compound of the invention include, but are not limited to Alzheimer's disease, Parkinson's disease, amyotrophic lateral sclerosis, epilepsy, astrocytoma, glioblastoma and Huntington's chorea.

Further examples of conditions which a compound of the invention may be used to prevent or treat include muscle impairment, fatigue and muscle fatigue. Muscle impairment and muscle fatigue may be prevented or treated in a healthy or compromised subject. A compromised subject may be, for instance, an individual suffering from myalgic encephalopathy (ME, or chronic fatigue syndrome) or the symptoms thereof. A compound of the invention may also be used to treat a patient suffering from a condition such as diabetes, metabolic syndrome X or hyperthyroidism, or a geriatric patient.

The aforementioned conditions are further examples of conditions which are caused by, exacerbated by or associated with elevated plasma levels of free fatty acids; the monoester compound of the invention can therefore be used to treat these conditions.

### Secondary Ester of (4R)-hydroxybutyl (3R)-hydroxybutyrate

### Composition of Matter

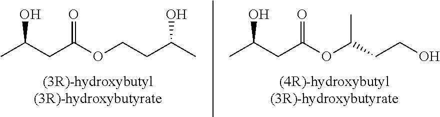

In reference to FIG. 1, our (4R)-hydroxybutyl (3R)-hydroxybutyrate “Secondary Ketone Ester” (SKE) can also be referred to as (2R)-4-hydroxybutan-2-yl (3R)-3-hydroxybutanoate. SKE, like the (3R)-hydroxybutyl (3R)-hydroxybutyrate (KME) in U.S. Pat. No. 8,642,654, will increase serum β-hydroxybutyrate compared to salts and the free acid of β-hydroxybutyrate when taken orally at the same dose. The mechanism of gastric degradation has been tested and elucidated by our team. Our dimer ketone ester (DE) wherein one molecule of β-hydroxybutyrate protects the first β-hydroxybutyrate at the beta hydroxy group will be even more stable in the gut than the KME or the SKE. The composition may also be administered via IV or other parenteral means.

Another iteration would be to protect the beta-hydroxy group U.S. Pat. No. 8,642,654 or free acid or ketone salt with an alcohol or polyhydroxy alcohol such as sorbitol, mannitol or xylitol or other non-toxic organic acids to prevent beta dehydration by forming an ester bond at the beta hydroxy group.

Although in low pH conditions, the exposed beta hydroxy groups of the SKE and KME dehydrate to a far lesser extent than the salts or free acid of β-hydroxybutyrate into a non-desirable compound which has no known or predicted metabolic benefit, the SKE and KME also can dehydrate. The Dimer Esters protecting the beta hydroxy group are far less susceptible than any other identified compound to dehydration because only one beta hydroxy group is exposed of the two effective molecules of β-hydroxybutyrate This is disclosed in the prior art providing room for more advantageous compounds and formulations thereof as described herein.

A trial comparing post oral administration serum levels of DbHB comprising a variety of ketone salts, show a considerable 30%-45% lower serum DbHB post ingestion when compared to ester in KME.

A clinical trial of the protected DEs are predicted to show an increase of I5%-30% serum DbHB in comparison to KME and 50%-75% increase when compared to an equivalent amount of salts of DbHB.

In addition to direct measurements of serum ketone levels with equal amounts of ketone salts and ketone esters, a second experiment was designed to prove the proposed mechanism of action to describe the mitigation of beta dehydration in the presence of strong acid or low pH environment by administration of an over the counter antacid prior to ingestion of ketone salts.

A third experiment was carried out exposing the salts and KME to conditions mimicking the pH of the gut. Rapid dehydration was observed yield undesirable products.

In summary, the DE provides an opportunity for greater efficacy while ingesting less of the active compound. This results in reduced gut irritation when compared to KME or other products utilized in the current arts.

The desired metabolic and extent of the therapeutic effects of taking any DbHB containing supplement or DbHB precursor supplements, are a function of serum levels of DbHB. Because any enantiomeric enriched ketone product including the salts, free acid, ketone esters and dimer ketone esters are very complicated to manufacture and very expensive, any method of increasing blood level efficacy and decreasing the amount of undesirable byproducts formed are very valuable. Our SKE, included with KME, will cut the effective cost by 30-45%, versus making near 100% KME. Our DE will further cut the effective cost by 50%-80% compared to KME, or SKE and KME blend, and 20-30% compared to hydroxybutyrate salts and free acid.

Our SKE and DEs are comprised of a diastereomer with very high enantiomeric excess GEE) with respect to the D enantiomer. Esters are typically synthesized by reacting an acid, in this case MB, with an alcohol, in the case of the SKE, 1,3-butanediol. The DE can be reacted with 1,3-butanediol, ethanol or any other non-toxic alcohol or sugar alcohol. Our bHB acid portions of the diastereomer esters are comprised of greater than 75-99% of the (D) enantiomer.

Our alcohol portion, when using chiral 1,3-butanediol in of either of the diastereomer esters or tristereomer DEs are comprised of a molecule with >85% (D) enantiomer. The final diastereomer and tristereomer esters have a very high total percentage of the (D) enantiomers. This is very important because the body can only utilize the (I)) enantiomer of MB as a source of fuel. Furthermore, D 1,3-butanediol is enzymatically oxidized into the free carboxylic acid DbHB in the liver. The higher the EE excess of the D 1,3-butanediol portion of our ester, the higher the serum level of DbHB shall be once converted by the liver into the free acid. The multi-step, protection/deprotection reaction and product has not been identified in a invivo ketone related biochemical environment and may not spontaneously happen, but can reliably be altered using the protection/deprotection steps.

This patent includes all analogous dimers and trimers using hydroxybutyrate and 1,3-butanediol and/or ethanol in addition to hydroxybutyrate.

Like KME, SKE will use the same enzyme which cleaves with water or hydrolyzes the diastereomers at the beta carbon. Those enzymes are found outside of the stomach in the small intestine and in the blood.

Another aspect that differentiates a bHB/bHB/1,3BD ester from KME or SKE is the fact that after hydrolysis only 1 molecule alcohol is released for every 2 molecules of β-hydroxybutyrate. The effective ratio of alcohol, after cleaving, to hydroxybutyrate is 1:2 compared to 1:1 for KME.

The benefits of these molecules would be to deliver the same total DbHB levels, without overloading the liver with greater alcohol.

### Therapeutic Applications

Intravenous DbHB has many uses as not only a primary fuel substrate via IV but an anti-inflammatory and neuro/myocardial protective agent indicated post acute stroke and heart attack. It is also indicated as the primary fuel substrate during any surgery wherein a source of fuel or calories is indicated. It's antioxidant properties and mechanism of action is very well described in the literature. The problem, until now, that has prevented the widespread use of DbHB during surgery and post stroke and heart attack is two fold. First, the cost of enantiomerically enriched free acid, salts or other analogs has been exceedingly high. Using racemic mixtures of bHB in any form is risky. The metabolic fate of the synthetic (L) enantiomer is unknown. The second problem is the risk of acidosis using the free acid or salt overload if using sodium, potassium, calcium magnesium salts. The ester prevents localized spikes in the acidity of the solution. Preferably a combination of partially buffered free acid using a combination of bufferering compounds using either/or sodium, potassium, calcium and magnesium together with the mono esters or DEs based on the natural ability to buffer the free acid and the ability to process salt making up the balance with esters in order to safely deliver 1-150 grams per day of β-hydroxybutyrate derived from any mixture of free acid, salts of β-hydroxybutyrate and esters of β-hydroxybutyrate.

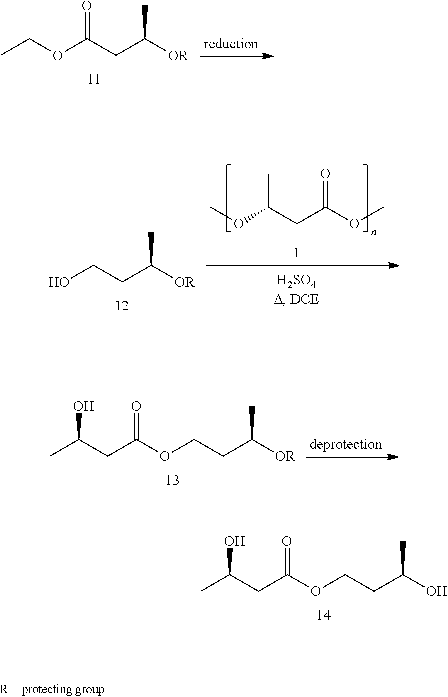

### Process

This compound is similar to the ketone ester (KME) identified in U.S. Pat. No. 8,642,654 and in the GRAS Notification. It is the product of the esterification of D 1,3-butanediol and D 3-hydroxybutyrate. But rather than making the ester bond between the primary #1 hydroxy group on butane diol, the ester bond is made with the secondary alcohol, referred to here as secondary ketone ester (SKE).

Ketosis has been shown to improve brain-function by providing a critical source of fuel to fuel starved cells due to a pathologically compromised inability to completely oxidize glucose. That pathologic inability is very likely at the root of many well-known neurodegenerative diseases such as Alzheimer's Disease, Parkinson's Disease and amyotrophic lateral sclerosis (ALS). The pathologic inability to process glucose is also very likely at the core of concussions and Traumatic Brain injury (TBI). The same inability is most likely at work in otherwise healthy adults who over time begin to exhibit problems with memory and other cognitive loss.

In addition to improved brain-function, ketones can improve muscle performance, such as in endurance athletes, and muscle recovery that would be beneficial to all athletes, including sprinters.

Aspects of recovery include greater protein synthesis after great physical exertion such as weight lifting, or sports. Aspects of the recovery include more rapid return to heart beat variability as a measure of the recovered post-exercise condition. Another aspect of the more rapid recovery includes heightened potentiating of the MTOR Complex (Mammalian Target of Rapamycin). Another aspect of faster recovery is between sets during exercise wherein a person is able to decrease the necessary rest time between sets. Also increased are the total repetitions possible before fatigue wherein many athletes have been able to achieve personal records for both the number of repetitions and maximum weight. Mood elevation effects also produce a greater willingness to engage in strenuous activity that otherwise may recover more encouragement.

Based on studies involving rats' hearts, Alzheimer's patients, and other studies, it may be shown that ketone concentrations in the blood above various threshold minima can provide therapeutic effects for a variety of neurological conditions such as Alzheimer's, Parkinson's, ALS, Multiple Sclerosis, traumatic brain injury, epilepsy, and autism, as well as non-neurological conditions such as diabetes types I & II. For example, (D)-β-hydroxybutyrate has been shown to act as a fuel substrate and substitute for glucose in diabetics as well as have hormone-like effects such as lowering of insulin levels.

Present embodiments can also be useful in treating hair loss, vision impairment, amyotrophic lateral sclerosis (commonly referred to as ALS or Lou Gehrig's disease), concussions, heart disease, diabetes, and traumatic brain injury, in addition to enhancing physical performance.

Present embodiments can also be useful in treating heart disease. The heart selectively takes up beta hydroxybutyrate over glucose when both substrates are available for use as a fuel. Myocardial output has been demonstrated to increase by an incredible 50% in healthy resting adults. The present embodiments should be indicated to improve myocardial output in all persons suffering from any myocardial impairment and in particular for persons waiting for a heart transplant to improve and prolong the lifespan while waiting.

Present embodiments can also be useful in treating acute stroke and post stroke recovery. In addition to ischemia, traumatized neurons also suffer from the glucose impairment resulting in insufficient fuel for homeostasis. The anti-inflammatory effects and general neuroprotective roles of hydroxybutyrate are well known.

### Formulation

In an example, the solid oral forms may contain, together with the active compound, diluents such as lactose, dextrose, saccharose, cellulose, corn starch or potato starch; lubricants such as silica, talc, stearic acid, magnesium or calcium stearate and/or polyethylene glycols; binding agents such as starches, arabic gum, gelatin, methyicellulose, carboxymethylcellulose, or polyvinyl pyrrolidone; disintegrating agents such as starch, alginic acid, alginates or sodium starch glycolate; effervescing mixtures; dyestuffs, sweeteners, wetting agents such as lecithin, polysorbates, lauryl sulphates, Such preparations may be manufactured in known manners, for example by means of mixing, granulating, tabletting, sugar coating, or film-coating processes.

Liquid dispersions for oral administration may be syrups, emulsions and suspensions. The syrups may contain as carrier, for example, saccharose or saccharose with glycerol and/or mannitol and/or sorbitol. In particular, a syrup for diabetic patients can contain as carriers only products, for example sorbitol or allulose, which do not metabolise to glucose or which only metabolise a very small amount to glucose. The suspensions and the emulsions may contain as carrier, for example, a natural gum, agar, sodium alginate, pectin, methylcellulose, carboxymethylcellulose or polyvinyl alcohol.

A compound of the invention as defined above is also suitably formulated into granules or a powder. In this form it can be readily dispersed in water or other liquid such as tea or a soft drink for human subjects to drink, for instance a beverage or drink as described above. It r ray also be encapsulated, tabletted or formulated with a physiologically acceptable vehicle into unit dosage forms. A unit dosage can comprise a therapeutically effective amount of the extract for a single daily administration, or it can be formulated into smaller quantities to provide for multiple doses in a day. The composition may thus, for instance, be formulated into tablets, capsules, syrups, elixirs, enteral formulations or any other orally administrable form.

Examples of physiologically acceptable carriers include water, oil, emulsions, alcohol or any other suitable material.

In addition to reducing plasma levels of fatty acids, a compound of the invention acts on the appetite centres in the brain. In particular, a compound of the invention increases the levels of various anorexigenic neuropeptides (neuropeptides known to be associated with decreased food intake and decreased appetites inthe appetite centres of the brain and also induces higher levels of malonyl CoA, a metabolite associated with decreased appetite and food intake. The invention therefore further provides a compound of the invention as defined above for use in treating a condition where weight loss or weight gain is implicated. For example, the compound may be used in suppressing appetite, treating obesity, promoting weight loss, maintaining a healthy weight or decreasing the ratio of fat to lean muscle in a subject. The subject n each case may be a healthy subject or a compromised subject. A healthy subject may be, for instance, an individual of healthy weight for whom physical performance and/or physical appearance is important. Examples include members of the military, athletes, bodybuilders and fashion models. A compromised subject may be an individual of non-healthy weight, for instance an individual who is overweight, clinically obese or clinically very obese. A compromised subject may alternatively be an individual of healthy or unhealthy weight who is suffering from a clinical Condition, for instance a condition listed below.

In addition to reducing plasma levels of fatty acids and acting on the appetite centre in the brain, a compound of the invention increases brain metabolic efficiency, by increasing brain phosphorylation potential and the ΔG′ of ATP hydrolysis. A compound of the invention thereby promotes improved cognitive function and can be used to treat cognitive dysfunction or to reduce the effects of neurodegeneration. A compound of the invention also increases the level of the neuropeptide Brain Derived Neurotrophic Factor (BDNF) in both the paraventricular nucleus (the appetite centre of the brain) and the hippocampus (a part of the brain known to be important for memory). As well as decreasing appetite, BDNF is known to prevent apoptosis and promote neuronal growth in basal ganglia and other areas of interest, thus the increased levels of BDNF produced by the compound of the invention are expected to inhibit neurodegeneration, limit neural tissue death after hypoxia or trauma and promote neural tissue growth.

A compound of the invention also increases the level of the anorexigenic neuropeptide Cocaine-and-Amphetamine Responsive Transcript (CART). CART is known to promote alertness as well as to decrease appetite. Thus, the increased levels of CART produced by the compound of the invention are expected to improve cognitive function.

The compounds of the invention are therefore useful for (a) promoting alertness and improved cognitive function, and (b) inhibiting neurodegeneration. The invention therefore further provides a compound of the invention as defined above for use in promoting alertness or improving cognitive function, or in treating cognitive dysfunction.

The invention also provides a compound of the invention as defined above for use in treating, preventing, or reducing the effects of, neurodegeneration, free radical toxicity, hypoxic conditions or hyperglycaemia.

In one embodiment, the compound of the invention as defined above is for use in treating, preventing, or reducing the effects of, neurodegeneration. A compound of the invention may be used to treat, prevent, or reduce the effects of neurodegeneration arising from any particular cause. The neurodegeneration may for instance be caused by a neurodegenerative disease or disorder, or may be caused by aging, trauma including stroke, anoxia and the like Examples of neurodegenerative diseases or disorders that can be treated using a compound of the invention include, but are not limited to Alzheimer's disease, Parkinson's disease, amyotrophic lateral sclerosis, epilepsy, astrocytoma, glioblastoma, and Huntington's chorea.

Further examples of conditions which a compound of the invention may be used to prevent or treat include muscle impairment, fatigue and muscle fatigue. Muscle impairment and muscle fatigue may be prevented or treated in a healthy or compromised subject. A compromised subject may be, for instance, an individual suffering from myalgic encephalopathy (ME, or chronic fatigue syndrome) or the symptoms thereof. A compound of the invention may also be used to treat a patient suffering from a condition such as diabetes, metabolic syndrome X or hyperthyroidism, or a geriatric patient.

The aforementioned conditions are further examples of conditions which are caused by, exacerbated by or associated with elevated plasma levels of free fatty acids; the monoester compound of the invention can therefore be used to treat these conditions.

In another embodiment, a compound of the invention is used to treat a patient suffering from a condition selected from diabetes, hyperpyrexia, hyperthyroidism, metabolic syndrome X, fever and infection, or a geriatric patient.

A compound of the invention may be administered in combination with one or more additional agents, for instance an agent selected from micronutrients and medicaments. The compound of the invention and the additional agent may be formulated together in a single composition for ingestion. Alternatively the compound of the invention and the additional agent may be formulated separately for separate, simultaneous or sequential administration.

When the additional agent is a medicament it may be, for instance, a standard therapy for a condition from which the subject is suffering. For instance, a compound of the invention may be administered in combination with conventional anti-diabetic agents to a subject suffering from diabetes. Conventional anti-diabetic agents include insulin sensitisers such as the thiazolidinedi ones, insulin secretagogues such as sulphonylureas, biguanide anti hyperglycemic agents such as metformin, and combinations thereof.

When the additional agent is a micronutrient it may be, for instance, a mineral, vitamin or antioxidant. Examples include iron, calcium, magnesium, vitamin A, the B vitamins, vitamin C, vitamin D and vitamin E.

The secondary ketone ester (SKE) may be useful by the body for a longer period of time versus KME. The molecule is cleaved by the body in order for it to be useful and provide beneficial effects. Because the bond may be harder to cleave in comparison to KME the active ingredient may be in the blood longer and the concentration curve in the blood may offer a broader time range of benefits to the consumer. The SKE may also offer more sustainable pH levels in the blood. A slower delivery of the SKE may also reduce spikes in pH of the blood common with other common ketone molecules in the arts.

As one skilled in the art will appreciate, embodiments of the present invention may be embodied as, among other things, a composition of matter and a method for making compositions of matter.

1.-6. (canceled)

7. A composition comprising (R)-4-hydroxybutan-2-yl (R)-3-hydroxybutanoate.

8. The composition of claim 7, further comprising (R)-3-hydroxybutyl (R)-3-hydroxybutanoate.

9. The composition of claim 8, further comprising one or more of the following:
a. β-hydroxybutyric acid/β-hydroxybutyric acid dimer;
b. β-hydroxybutyric acid/1,3-butanediol/β-hydroxybutyric acid trimer; and
c. β-hydroxybutyric acid/β-hydroxybutyric acid/1,3-butanediol trimer.

10. The composition of claim 9, further comprising one or more of the following:
a. β-hydroxybutyric acid salts;
b. β-hydroxybutyric acid;
c. 1,3 butanediol; and
d. MCT oil.

11. The composition of claim 10, having at least 60% enantiomeric excess of any of the following:
a. (R)-4-hydroxybutan-2-yl(R)-3-hydroxybutanoate;
b. (R)-3-hydroxybutyl(R)-3-hydroxybutanoate;
c. β-(R)-hydroxybutyric acid/β-(R)-hydroxybutyric acid dimer;
d. β-(R)-hydroxybutyric acid salts;
e. β-(R)-hydroxybutyric acid;
f. (R)-1,3 butanediol;
g. β-(R)-hydroxybutyric acid/(R)-1,3-butanediol/β-(R)-hydroxybutyric acid trimer; and
h. β-(R)-hydroxybutyric acid/β-(R)-hydroxybutyric acid/(R)-1,3-butanediol trimer.
